(19) United States
(12) Patent Application Publication
(10) Pub. No.: US 2010/0113378 A1
(43) Pub. Date: May 6, 2010

(54) Compositions and Methods for Treating and Preventing Cancer Using Analogs of Vitamin D

(76) Inventors: Rahul Ray, Wayland, MA (US); Narashima Swamy, Providence, RI (US)

Correspondence Address: BURNS & LEVINSON, LLP, 125 SUMMER STREET, BOSTON, MA, 02110, US

(21) Appl. No.: 12/646,605

(22) Filed: Dec. 23, 2009

Related U.S. Application Data
(63) Continuation of application No. 12/197,923, filed on Aug. 25, 2008, now abandoned.
Application No. 12/197,923 is a continuation of application No. 10/870,122, filed on Jun. 17, 2004, now abandoned.
(60) Provisional application No. 60/479,128, filed on Jun. 17, 2003.

(51) Int. Cl. A61K 31/704; C07C 401/00; A61K 31/593; A61K 31/66; A61P 35/00

(52) U.S. Cl. 514/34; 552/653; 514/167; 514/110

(57) ABSTRACT

Disclosed are compositions and methods used to treat and/or prevent cancer and metabolic diseases, such as psoriasis. In one aspect, the present invention pertains to the use of cross-linking analogs of vitamin D and its metabolites employed for the treatment of prostate cancer.

### RELATED APPLICATIONS

This application claims priority to and the benefit of U.S. Provisional Application 60/479,128, filed Jun. 17, 2003.

### GOVERNMENTAL SUPPORT

This invention was made with United States government support under Contract Number DK 47418 awarded by the National Institutes of Health. The Government has certain rights in this invention.

### FIELD OF THE INVENTION

The present invention pertains to the use of cross-linking analogs of vitamin D and its metabolites employed for the treatment of prostate cancer.

### BACKGROUND OF THE INVENTION

Prostate cancer is the leading cause of cancer-death among American males. Approximately 10 million men in the United States are currently diagnosed with prostate cancer, and the number is on the rise. Several epidemiological studies have identified age, race and geography as major risk factors for prostate cancer. The risk of prostate cancer increases with age; and greater than 80% incidence are found in men of age 65 and above.

Geography plays a significant role in prostate cancer. For example, prostate cancer mortality rate is higher among Caucasians in countries in the Northern hemisphere compared to those in the Southern hemisphere. Prostate cancer is rare in sub-Saharan Africa, but common among African Americans. African Americans are also at a greater risk than Americans of Caucasian origin.

The above-mentioned findings strongly suggest a correlation between prostate cancer and exposure to sun. Sunlight is an essential ingredient in the cutaneous biosynthesis of vitamin D, an essential nutrient (see, FIG. 1). In the United States, vitamin D is also supplemented in milk. However, for the elderly, full-body exposure to the sun is severely restricted; and lactose-intolerance is common. For dark-skinned people, skin melanin substantially decreases the production of vitamin D. To aggravate the matter further, low levels of vitamin D have been implicated in the predisposition for the development of cancers in many organs and tissues including prostate.

Currently, there exists a need to effectively treat or prevent the onset of prostate cancer. The invention disclosed herein describes an effective regime that can be utilized in treating or preventing prostate cancer.

### BRIEF SUMMARY OF THE INVENTION

The current invention is direct to compositions and methods used to treat and/or prevent cancer and metabolic diseases, such as psoriasis. In one aspect, the present invention pertains to the use of cross-linking analogs of vitamin D and its metabolites employed for the treatment of prostate cancer.

One embodiment of the present invention is directed to analogs of 1,25(OH)₂D₃ and its metabolites and derivatives. In one aspect, the analog of 1,25(OH)₂D₃ cross links 1,25(OH)₂D₃ to the hormone-binding pocket of VDR. (It is important to note that 1,25(OH)₂D₃ incorporates 1,25(OH)₂D₂ and 1,25(OH)₂D₅, therefore, when 1,25(OH)₂D₃ is mentioned, D₂ and D₅ are to be understood as being included.) In one aspect, the analog is 1,25(OH)₂D₃-3-BE, or a derivative thereof. In another aspect, the analog is 25(OH)₂D₃-3-BE, or a derivative thereof.

In another embodiment, the invention is directed to methods of treating and/or preventing cancer in a subject by administering an effective amount of an analog of 1,25(OH)₂D₃. In one aspect, the analog to be administered is 1,25(OH)₂D₃-3-BE. In another aspect, the analog to be administered is 25(OH)₂D₃-3-BE.

In another embodiment, the invention is directed to the treatment and/or prevention of cancer using combination therapy. In this embodiment, a subject is administered a combination of an effective amount of an analog of 1,25(OH)₂D₃. In one aspect, the analog to be administered is 1,25(OH)₂D₃-3-BE together with a known oncolytic agent. The administration of both components can be simultaneously, or in tandem.

For a better understanding of the present invention, together with other and further objects thereof, reference is made to the accompanying drawings and detailed description and its scope will be pointed out in the appended claims.

### BRIEF DESCRIPTION OF THE SEVERAL VIEWS OF THE DRAWING

FIG. 1 shows biosynthesis and receptor mediated actions of 1,25(OH)₂D₃;

FIG. 2 shows cross-linking of 1,25 (OH)₂D₃-3-BE, CL analog) to the hormone-binding pocket of VDR via Cys288;

FIG. 3 shows the structure of Vitamin D₂, D₃, and D₅ derivatives;

FIG. 4 shows the effects of 1,25(OH)₂D₃ and 1,25(OH)₂D₃-3-BE on the proliferation of keratinocytes, MCF-7, PC-3, LNCaP, and PZ-HPV-7 cells;

FIG. 5 shows the microscopic appearance of LNCaP cells 16 hours after treatment with 1,25(OH)₂D₃-3-BE;

FIG. 6 shows the effects of 1,25(OH)₂D₃ and 1,25(OH)₂D₃-3-BE on viable cell-count and anti-proliferation in LNCaP cells;

FIG. 7 shows the effects of 1,25(OH)₂D₃ and 1,25(OH)₂D₃-3-BE on viable cell-count and anti-proliferation in PZ-HPV-7 cells;

FIG. 8 shows the effect of different doses of 1,25(OH)₂D₃ or 1,25(OH)₂D₃-3-BE on the proliferation of LNCaP cells;

FIG. 9 shows the effect of different doses of 1,25(OH)₂D₃ or 1,25(OH)₂D₃-3-BE on LNCaP viable cell-count;

FIG. 10 shows the hydrolysis of the ester bond to produce bromoacetic acid;

FIG. 11 shows the effects of 1,25(OH)₂D₃, 1,25(OH)₂D₃-3-BE, and bromoacetic acid on the proliferation of MCF-7 and PC3 cells;

FIG. 12a shows the structure of 1,25(OH)₂D₃-3-BE;

FIG. 12b shows the structure of 25-OH-D₃-3-BE;

FIG. 13 shows that 25-hydroxyvitamin D₃-2-bromoacetate (25-OH-D₃-3-BE) specifically cross-links to the hormone binding pocket of VDR;

FIG. 14 shows ³H-Thymidine incorporation assays of 25-OH-D₃-3-BE with PZ-HPV-7 cells;

FIG. 15 shows ³H-Thymidine incorporation assays of 25-OH-D₃-3-BE with keratinocytes;

FIG. 16 shows ³H-Thymidine incorporation assays of 25-OH-D₃-3-BE with LNCaP cells;

FIG. 17 shows ³H-Thymidine incorporation assays of 25-OH-D₃-3-BE with PC-3 cells;

FIG. 18 shows ³H-Thymidine incorporation assays of 25-OH-D₃-3-BE with PC-3 cells;

FIG. 19 shows ³H-Thymidine incorporation assays of 25-OH-D₃-3-BE with MCF-7 cells;

FIG. 20 shows H&E staining of tumor sections. FIG. 20a shows the control of sesame oil; and FIG. 20b shows the section treated with 25-OH-D₃-3-BE;

FIG. 21 shows hydrolysis of 25-OH-D₃-3-BE to produce 25-OH-D₃ and bromoacetic acid;

FIG. 22 shows ³H-Thymidine incorporation assays of bromoacetic acid (10⁶) and 25-OH-D₃-3-BE (10⁻⁶M) with PC-3 cells;

FIG. 23 shows ³H-Thymidine incorporation assays of 25-OH-D₃-3-BE (10⁻⁶M), and 25-OH-D₃-3-BE (10⁻⁶M) with bromoacetic acid with PC-3 cells;

FIG. 24 shows DNA fragmentation analysis of PC-3 cells; and

FIG. 25 shows capase activity in PC-3 cells treated with 1,25(OH)₂D₃, 25-OH-D₃, or 25-OH-D₃-3-BE.

### DETAILED DESCRIPTION OF THE INVENTION

The current invention is direct to compositions and methods used to treat and/or prevent cancer and metabolic diseases, such as psoriasis. In one aspect, the present invention pertains to the use of cross-linking analogs of vitamin D and its metabolites employed for the treatment of prostate cancer.

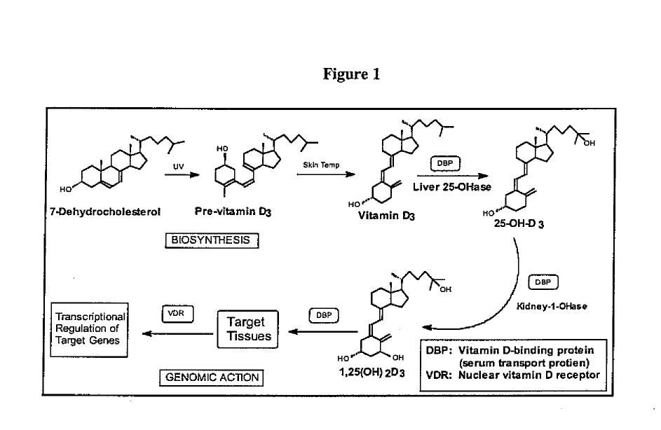

Vitamin D is biologically inactive, but can be activated by two biological oxidations to produce 1-α,25-dihydroxyvitamin D₃ (1,25(OH)₂D₃, Calcitriol), the hormonally active form of vitamin D₃. Similar to other steroid hormones such as estrogen, progesterone, glucocorticoids etc., biological activity of 1,25(OH)₂D₃ is manifested by its high-affinity binding to its nuclear receptor (vitamin D receptor, VDR). The hormone-bound VDR further binds to another nuclear protein (RXR), and the ternary complex interacts with the vitamin D-response element in the promoter region of the vitamin D-controlled genes to activate transcription and translation (Norman A W, Receptors for 1α,25(OH)₂D₃ (1998) J. Bone & Min. Res., 13 1360-1369; Ray R. Molecular recognition and structure-activity relations of the vitamin D-binding protein and vitamin D receptor. In: Vitamin D: Physiology, Molecular Biology and Clinical Applications. Editor, M. F. Holick, Humana Press, NJ, pp 147-162 (1999), the entire teachings of which are incorporate herein by reference). Biosynthesis of 1,25(OH)₂D₃ and the role of VDR are shown in FIG. 1.

The 1,25(OH)₂D₃ is a pluripotent hormone with properties that include calcium and phosphorus-homeostasis, regulation of the growth and maturity of cells, and modulation of the immune system (Jones G, Strugnell S A, DeLuca H F. Current understanding of the molecular actions of vitamin D. (1998) Physiol Rev 78:1193-1231, the entire teaching of which is incorporated herein). It is well-established that 1,25(OH)₂D₃ is antiproliferative and pro-differentiative in vitro and in vivo in numerous malignant cells, including prostate cancer, and metabolic diseases, such as psoriasis (Bouillon R, Okamura W H, and Norman A W. Structure-function relationships in the vitamin D endocrine system. (1995) Endocr Rev 16:200-257; Schwartz G G, Hill C, Oder T A, Becich M J & Bahnson R R 1,25-dihydroxy-16-ene-23-yne-vitamin D₃ and prostate cancer cell proliferation in vivo. (1995) Urology 46 365-369; Skowronski R J, Peehl D M & Feldman D. Actions of vitamin D₃ analogs on human prostate cancer cell lines: comparison with 1,25-dihydroxyvitamin D₃. (1995) Endocrinology 136 20-26; Campbell M J & Koeffler H P. Toward therapeutic intervention of cancer by vitamin D compounds. (1997) Journal of the National Cancer Institute 89 182-185, the entire teachings of which are incorporated herein). Efficacy of 1,25(OH)₂D₃ and its analogs in the treatment of prostate tumor has been shown by several investigative teams in vitro as well as in vivo. For example Johnson and Trump et al. have demonstrated that 1,25(OH)₂D₃ and its analogs, Ro23-7553, Ro25-6760 and EB1089 have significant anti-tumor activity in the treatment of established tumors, prevention of tumor outgrowth and in decreasing the number and size of metastases in human xenograft prostatic adenocarcinoma (PC-3) and the Dunning rat metastatic prostatic adenocarcinoma (Getzenberg R H, Light B W, Lapco P E, Konety B R, Nangia A K, Acierno J S, Dhir R, Shurin Z, Day R S, Trump D L, Johnson C S. Vitamin D inhibition of prostate adenocarcinoma growth and metastasis in the Dunning rat prostate model system. (1997) Urology 50:999-1006, the entire teaching of which is incorporated herein). Furthermore, they observed that 1,25(OH)₂D₃, Ro23-7553 and EB1089 potentiated the cytotoxic activity of cisplatin, carboplatin, paclitaxel and docetaxel in prostate-tumor (Light B W, Yu W D, McElwain M C, Russell D M, Trump D L, Johnson C S. Potentiation of cisplatin antitumor activity using a vitamin D analogue in a murine squamous cell carcinoma model system. (1997) Cancer Res. 57:3759-3764, the entire teaching of which is incorporated herein), while dexamethasone potentiated the anti-tumor effect of 1,25(OH)₂D₃ and decreased 1,25(OH)₂D₃-induced hypercalcemia (Yu W D, McElwain M C, Modzelewski R A, Russell D M, Smith D C, Trump D L, Johnson C S. Enhancement of 1,25-dihydroxyvitamin D3-mediated antitumor activity with dexamethasone. (1998) J Natl Cancer Inst. 90:134-141, the entire teaching of which is incorporated herein). However, the need for 1,25(OH)₂D₃-analogs with low toxicity and tissue-specific activity has remained unabated.

In the past, toxicity resulting from hypercalcemia is a side-effect commonly associated with 1,25(OH)₂D₃ and some of its analogs, particularly at clinically required high dose-levels. Another problem has involved the lack of tissue-specificity for drug action—a similar concern with other cancer drugs. This lack of target-specificity demands the use of a high dose of these drugs with concomitant increase in toxicity. Furthermore, 1,25(OH)₂D₃ and some of its analogs are known to be antiproliferative towards cancer cells, but not cytotoxic, particularly at dose levels that do not induce hypercalcemia. Hence when the drug is withdrawn, malignancy returns.

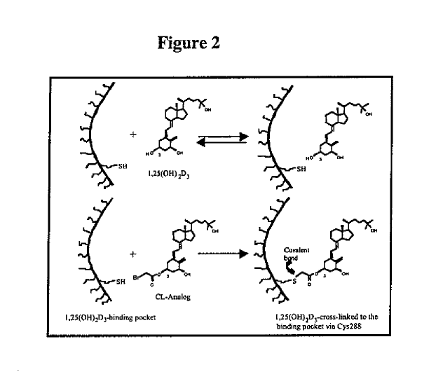

The present invention is directed, in part, to novel analogs of 1,25(OH)₂D₃, for example, 1,25(OH)₂D₃-3-BE and 25(OH)₂D₃-3-BE. The inventors have developed a novel affinity alkylating analog of 1,25(OH)₂D₃ which cross links 1,25(OH)₂D₃ to the hormone-binding pocket of VDR via a nucleophilic displacement reaction between a Cys residue in the hormone-binding pocket (Cys288) and a reactive bromoacetate group at the 3-position of 1,25(OH)₂D₃ (Ray R, Ray S., and Holick M. F. 1″,25-dihydroxyvitamin D₃-3-deoxy-3$-bromoacetate, an affinity labeling analog of 1″,25-dihydroxyvitamin D₃. (1994) Bioorg. Chem. 22 276-283; Ray R, Swamy N, MacDonald P N, Ray S, Haussler M R, and Holick M F. (1996) Affinity labeling of 1″,25-dihydroxyvitamin D₃ receptor. J. Biol Chem 271 2012-2017; Swamy N, Kounine M, and Ray R. (1997) Identification of the subdomain in the nuclear receptor for the hormonal form of vitamin D₃, 1″,25-dihydroxyvitamin D₃, vitamin D receptor, that is covalently modified by an affinity labeling reagent. Arch Biochem Biophys 348 91-95; Chen M L, Ray S, Swamy N, Holick M F, and Ray R. Mechanistic studies to evaluate the enhanced anti-proliferation of human keratinocytes by 1α,25-dihydroxyvitamin D₃-3-bromoacetate, a covalent modifier of vitamin D receptor, compared to 1α,25-dihydroxyvitamin D₃. (1999) Arch. Biochem. Biophys. 370 34-44; Swamy N, Xu W, Paz N, Hsieh J-C, Haussler M R, Maalouf G J, Mohr S C, & Ray R. Molecular modeling, affinity labeling and site-directed mutagenesis define the key points of interaction between the ligand-binding domain of the vitamin D nuclear receptor and 1,25-dihydroxyvitamin D₃. (2000) Biochemistry 39: 12162-12171, the entire teachings of which are incorporated herein), as shown in FIG. 2. It should be emphasized that 1,25(OH)₂D₃ also binds to the same binding pocket of VDR, however, the binding is non-covalent. Furthermore, binding of 1,25(OH)₂D₃ to VDR is an equilibrium process, while that of 1,25(OH)₂D₃-3-BE is a non-equilibrium process.

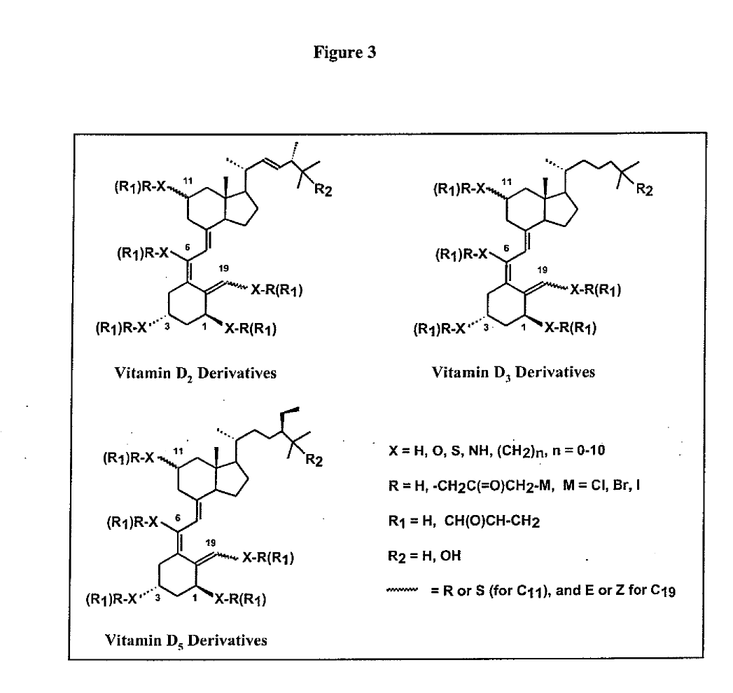

In one aspect of the present invention, similar α-halocarbonyl (halo=chloro, bromo, iodo; carbonyl=ester, ketone, amide) as well as epoxide derivatives of 1,25(OH)₂D₃ and 25-OH-D₃ and other metabolites of vitamin D also have unique tissue-specific and cancer cell-killing properties. In addition, these analogs can carry the α-halocarbonyl groups at different positions of the parent vitamin D molecule, as well as other vitamin D related molecules such as vitamin D₂ and vitamin D₅ and their metabolites, as shown in FIG. 3.

It has been demonstrated that the cross-linking of this analog (i.e., 1,25(OH)₂D₃-3-BE) to VDR is extremely rapid and resulted in an increased transcription of VDR-regulated genes, and sustained a higher level of transcription for a longer duration at a lower concentration of the analog (Chen M L, Ray S, Swamy N, Holick M F, and Ray R. Mechanistic studies to evaluate the enhanced anti-proliferation of human keratinocytes by 1α,25-dihydroxyvitamin D₃-3-bromoacetate, a covalent modifier of vitamin D receptor, compared to 1α,25-dihydroxyvitamin D₃. (1999) Arch. Biochem. Biophys. 370 34-44, the entire teaching of which is incorporated herein). Additionally, 1,25(OH)₂D₃-3-BE was found to have a lower calcemic index than 1,25(OH)₂D₃ in intestinal cancer Caco-2 cells (Van Auken M, Buckley D, Ray R, Holick M F and Baran D. Effects of the vitamin D₃ analog 1″,25-dihydroxyvitamin D₃-3$-bromoacetate on rat osteosarcoma cells: comparison with 1″,25-dihydroxyvitamin D₃. (1996) Journal of Cellular Biochemistry 63 302-310, the entire teaching of which is incorporated herein).

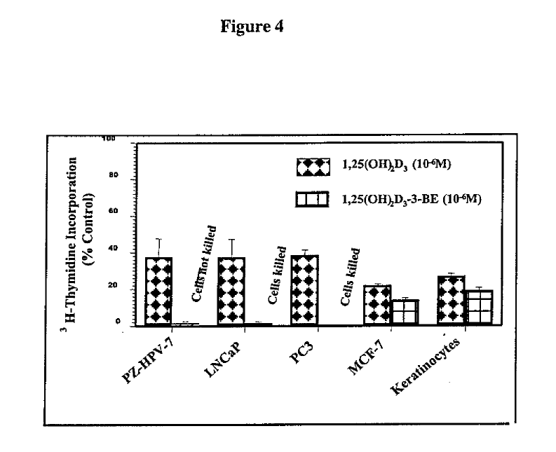

It is well known that secretory epithelial cells are the main sites for the development of cancers of the breast, prostate and various other organs. Hence, in a preliminary study, the inventors compared the growth-inhibitory effects of 1,25(OH)₂D₃-3-BE in VDR positive epithelial cells; i.e., keratinocytes (primary cells), MCF-7 cells (breast cancer cells), LNCaP and PC3 cells (prostate cancer cells), and PZ-HPV-7 (papillovirus immortalized normal prostate cells) by using classical ³H-thymidine incorporation assays. Referring to FIG. 4, results of these assays demonstrated that 1,25(OH)₂D₃-3-BE has a stronger antiproliferafive effect on all the cells compared to 1,25(OH)₂D₃. However, this effect was most pronounced in prostate cells.

Furthermore, microscopic examination of the cells showed that, when treated with 1,25(OH)₂D₃-3-BE for sixteen (16) hours, a majority of hormone-sensitive LNCaP and hormone-refractory PC3 cells appeared to have undergone apoptosis, indicating that 1,25(OH)₂D₃-3-BE may be cytotoxic. In addition, such observation was limited only to prostate cancer cells, because immortalized normal prostate cells (PZ-HPV-7), breast cancer cells (MCF-7) or primary cultures of normal human keratinocytes did not show any such behavior and appeared normal (although reduced in number). Furthermore, all the cells treated with 1,25(OH)₂D₃ appeared normal (see, FIG. 5).

The inventors further studied the effect of 1,25(OH)₂D₃-3-BE on LNCaP cells (most closely resembling human hormone-sensitive prostate cancer) and PZ-HPV-7 cells (immortalized normal prostate cells). The cells were treated with ethanol or 10⁻⁶M of either 1,25(OH)₂D₃ or 1,25(OH)₂D₃-3-BE for 16 hours and the effect on proliferation was determined by ³H-thymidine incorporation assays as described earlier. The viable cell count was determined by Methylene blue assay. The Methylene blue assay is a simple colorimetric method that is widely used to determine the viable cell count.

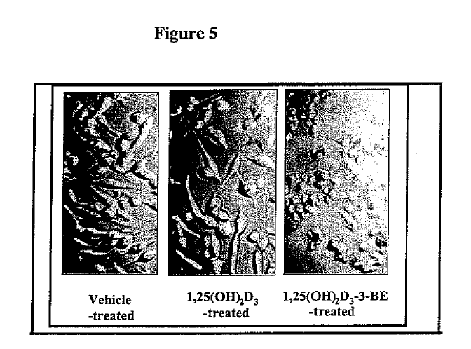

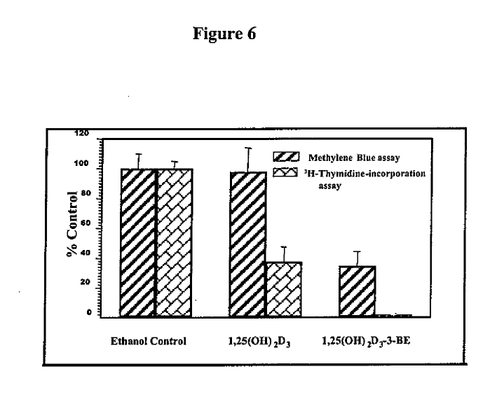

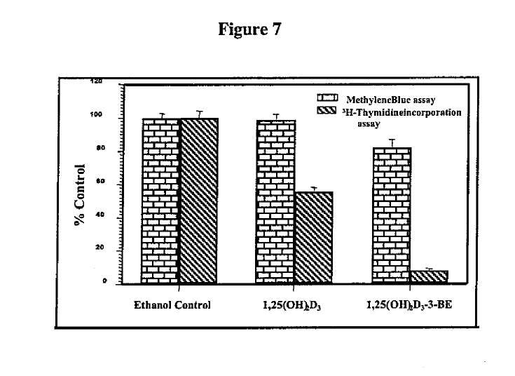

Referring to FIGS. 6 & 7, results of these assays demonstrated that 1,25(OH)₂D₃ showed only inhibition of proliferation, but no cytotoxicity on either LNCaP or PZ-HPV-7 cells; and viable cell counts, determined by Methylene blue assay, was similar to that of the control. However, the effect of 1,25(OH)₂D₃-3-BE was strikingly different on PZ-HPV-7 cells when compared to LNCaP cells. Although proliferation was almost completely inhibited in the case of both LNCaP and PZ-HPV-7 cells, the viable cell count was similar to control only in the case of PZ-HPV-7 (diminished by approximately 15%). In contrast, the viable cell count was diminished by approximately 60% in case of LNCaP cells. These results emphasized that the cytotoxic effects of 1,25(OH)₂D₃-3-BE may be specific for prostate cancer cells; and normal prostate cells may not be affected significantly (i.e., by 1,25(OH)₂D₃-3-BE).

The inventors studied dose-dependency of antiproliferation and cytotoxicity by 1,25(OH)₂D₃ or 1,25(OH)₂D₃-3-BE in LNCaP cells. The cells were treated with 10⁻⁸-10⁻⁶M of either 1,25(OH)₂D₃ or 1,25(OH)₂D₃-3-BE or ethanol (used as a control). The effect on proliferation and viable cell count were determined by using ³H-thymidine incorporation and Methylene blue assay respectively.

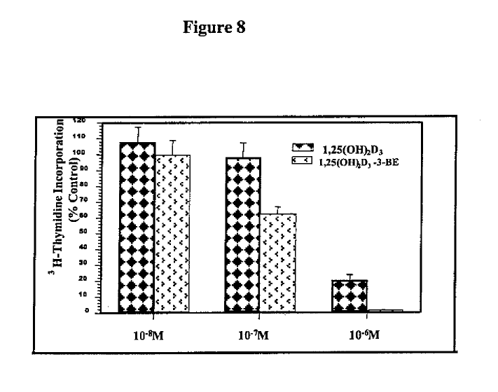

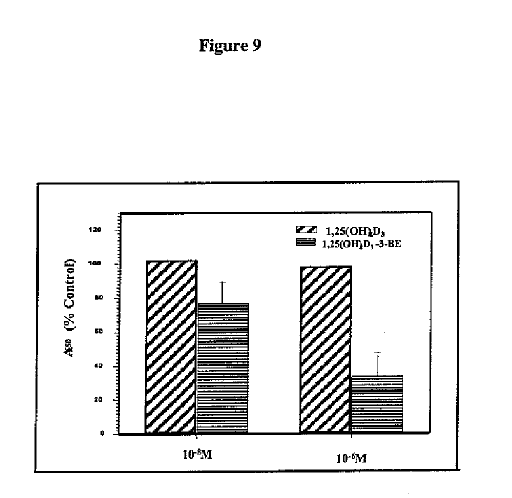

Results of these assays, shown in FIGS. 8 and 9, demonstrated that antiproliferative effects of 1,25(OH)₂D₃ and 1,25(OH)₂D₃-3-BE in LNCaP cells were most pronounced at 10⁻⁶M; further substantiating the results obtained by others (Schwartz G G, Hill C, Oeler T A, Becich M J & Bahnson R R 1,25-dihydroxy-16-ene-23-yne-vitamin D₃ and prostate cancer cell proliferation in vivo. (1995) Urology 46 365-369; Skowronski Peehl D M & Feldman D. Actions of vitamin D₃ analogs on human prostate cancer cell lines: comparison with 1,25-dihydroxyvitamin D₃. (1995) Endocrinology 136 20-26, the entire teachings of which are incorporated herein). However, 1,25(OH)₂D₃-3-BE was much stronger in decreasing the proliferation than 1,25(OH)₂D₃ at dose levels of 10⁻⁶ and 10⁻⁷M.

Results of the above experiments are summarized below:
1,25(OH)₂D₃ and 1,25(OH)₂D₃-3-BE showed dose-dependant antiproliferation in kertinocytes, MCF-7 LNCaP, PC3 and PZ-HPV-7 cells;
1,25(OH)₂D₃-3-BE was a stronger antiproliferative agent than 1,25(OH)₂D₃ at every dose level;
1,25(OH)₂D₃-3-BE preferentially inhibited the growth of prostate cell types;
1,25(OH)₂D₃-3-BE was cytotoxic only to prostate cancer cells displaying a tissue specificity; and
1,25(OH)₂D₃-3-BE was cytotoxic only to prostate cancer cells and not to normal prostate cells.

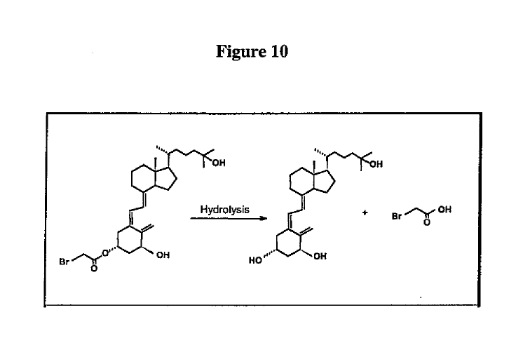

Although results in preliminary studies were promising, the inventors were concerned that in the cellular assays hydrolysis of the ester bond in 1,25(OH)₂D₃-3-BE might produce 1,25(OH)₂D₃ and bromoacetic acid (as shown in FIG. 10); and bromoacetaic acid might cross-link to proteins randomly to produce the observed effects.

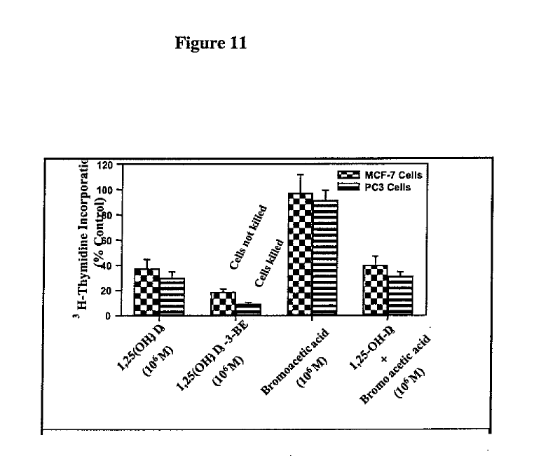

Antiproliferation assays of 1,25(OH)₂D₃, 1,25(OH)₂D₃-3-BE, bromoacetaic acid and a mixture of equimolar amounts of 1,25(OH)₂D₃ and bromoacetic acid in MCF-7 and LNCaP cells demonstrated that bromoacetic acid alone had no effect on these cells; and a mixture of 1,25(OH)₂D₃ and bromoacetic acid produced same results as obtained with 1,25(OH)₂D₃ alone (FIG. 11). These results strongly suggested that 1,25(OH)₂D₃-3-BE as an intact molecule is responsible for its observed antiproliferative effect in LNCaP and MCF-7 cells.

It is well-established that the biological properties of 1,25(OH)₂D₃ and its analogs are mediated by their interaction with VDR. In order to establish that the observed antiproliferative property of 1,25(OH)₂D₃-BE is also manifested via VDR, investigators carried out antiproliferation assays of 1,25(OH)₂D₃, 1,25(OH)₂D₃-3-BE and benzylbormoacetate, a non-vitamin D protein alkylating agent. Results of these assays (not shown) demonstrated that benzylbormoacetate had no effect (proliferative or antiproliferative) on LNCaP and MCF-7 cells.

Collectively, results from the experiments described above strongly indicate that 1,25(OH)₂D₃-3-BE, in its intact molecular form, interacts with nuclear VDR to elicit cytostatic and cytotoxic behavior towards prostate cancer cells.

A serious concern in the use of 1,25(OH)₂D₃ and some its analogs (not those claimed in the present invention) for therapy involves toxicity of the parent hormone, particularly at clinically required high dose-levels. In contrast, 25-hydroxyvitamin D₃ (25-OH-D₃), the immediate metabolic precursor of 1,25(OH)₂D₃ (see, FIG. 1) is known to be non-toxic (serum-level of 25-OH-D₃ is approximately 1000-fold less than that of 1,25(OH)₂D₃). However, 25-OH-D₃ or any of its synthetic analogs have not been seriously considered as alternative low-toxicity anti-cancer agents because 25-OH-D₃ is known to possess nominal biological effects due to its significantly low VDR-binding ability compared with 1,25(OH)₂D₃, the parent hormone.

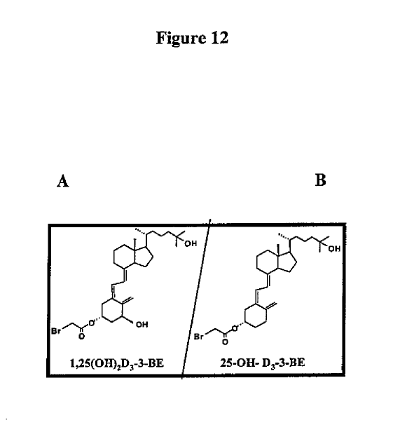

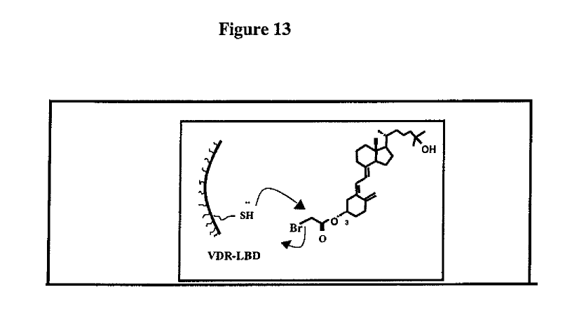

Without wishing to be bound by theory, one possible model is that 25-hydroxyvitamin D₃-3-bromoacetate (25-OH-D₃-3-BE) which is similar to 1,25-dihydroxyvitamin D₃-3-bromoacetate (1,25(OH)₂D₃-3-BE) without the 1-hydroxyl group, might bind to VDR with low affinity (see, FIGS. 12A, 12B). But due to the kinetic nature of the process, ultimately all of this compound should covalently attach to the hormone-binding pocket (of VDR). Investigators recently proved this hypothesis by demonstrating that 25-OH-D₃-3-BE specifically labels the hormone-binding pocket of VDR (N. Swamy, J. Addo, and R. Ray. Development of an affinity-driven double cross-linker: isolation of a ligand-activated factor, associated with vitamin D receptor-mediated transcriptional machinery. (2000) Bioorganic and Medicinal Chemistry Letters 10: 361-364, the teaching of which is incorporated herein) (as shown in FIG. 13).

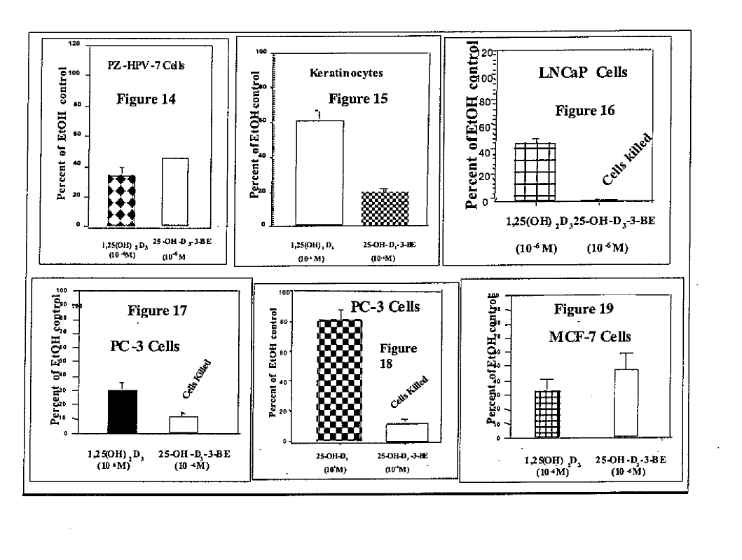

In a study, investigators compared the growth-inhibitory effects of 25-OH-D₃-3-BE and 1,25(OH)₂D₃ in several VDR-positive epithelial cells, e.g. keratinocytes (primary skin cells), MCF-7 cells (breast cancer cells), hormone-sensitive LNCaP and hormone-refractory PC3 cells (prostate cancer cells), and PZ-HPV-7 (papillovirus immortalized normal prostate cells) by using ³H-thymidine incorporation assays (FIGS. 14-19). Results of these assays showed that: (a) 10⁻⁶M of 25-OH-D₃-3-BE, despite being a derivative of 25-OH-D₃, was antiproliferative to all the cells tested, and it was a stronger antiproliferative agent than 1,25(OH)₂D₃ in LNCaP and PC-3 cells; and (b) 10⁻⁶M of 25-OH-D₃-3-BE was lethal to LNCaP (FIG. 16) and PC-3 cells (FIGS. 17, 18), but not to immortalized normal prostate cells (PZ-HPV-7 cells) (FIG. 14), keratinocytes (FIG. 15) and MCF-7 cells (FIG. 19).

Collectively, results from these experiments demonstrate that 25-OH-D₃-3-BE can be developed as a strong antiproliferative and cytotoxic agent for prostate cancer, specifically hormone-sensitive, hormone refractory, and metastatic cancers.

The dose of 25-OH-D₃-3-BE that induced strong antiproliferation and cytotoxicity (in PC-3 and LNCaP cells) is high, and such a dose of 1,25(OH)₂D₃ and many of its analogs are known to induce toxicity in animals. However, an analog of 25-OH-D₃11,25(OH)₂D₃ could be useful in micromolar concentration as long as it does not show systemic toxicity.

Investigators completed an in vivo study to determine the toxicity of 25-OH-D₃-3-BE at various doses in CD-1 mice. The animals (average weight ˜30 gms) were maintained with normal chow and water ad libitum. Different doses of either 25-OH-D₃ (in saline, 3.3 or 33 μg/kg), or 25-OH-D₃-3-BE (in saline, 3.3, 33 or 166.7 μg/kg) or saline control (0.2 ml) were administered to these animals (in groups of three) intraperitoneally over a period of three weeks. At the end of the experiment the animals were lightly anesthetized and blood collected after decapitation for serum calcium-analysis. During the entire experiment animals were observed for any sign of toxicity i.e. lethargy, loss of appetite, loss of weight etc. Frequency of administration, body weights at the beginning and at the end of the experiment and serum calcium values are given in the accompanying Table 1.

TABLE 1

| | Body Weight | Body Weight | Serum Calcium |
|---|---|---|---|
| | On Day 0 | on Day 12 | (mg/ml) |
| | (grams) | (grams) | on day 12 |
| Saline Control | 29.6 ± 2.4 | 30.6 ± 2.7 | 9.23 ± 0.15 |
| 25-OH-D₃ | 31.5 ± 1.56 | 33.5 ± 1 | 9.6 ± 0.2 |
| (3.3 μg/kg) | | | |
| 25-OH-D₃ | 28.9 ± 2 | 30.66 ± 2 | 9.7 ± 0.8 |
| (33 μg/kg) | | | |
| 25-OH-D₃-3-BE | 29 ± 0.6 | 30.36 ± 1 | 9.1 ± 0.2 |
| (3.3 μg/kg) | | | |
| 25-OH-D₃-3-BE | 31 ± 0.83 | 32.9 ± 1.4 | 9.4 ± 0.2 |
| (33 μg/kg) | | | |
| 25-OH-D₃-3-BE | 30.55 ± 0.7 | 30.7 ± 1 | 9.7 ± 0.1 |
| (166.7 μg/kg) | | | |

According to the above Table 1,25-OH-D₃-3-BE is non-toxic in vivo at a dose as high as 166.7 μg/Kg in a mouse, but indications are that much higher doses can be employed.

The in vivo effect of 25-OH-D₃-3-BE in nude mice bearing prostate tumor induced with PC-3 cells was examined.

Prostate tumor was induced in male SCID mice (Jackson Labs, average weight 20 gm, group of three) by s.c. inoculation of PC-3 cells grown in DMEM media with 10% FCS (10⁶ cells, 1:1 with Matrigel) until the tumors had an average size of approximately 150 mm³. Sesame oil solutions (200 μl) of either 25-OH-D₃ (10 μg) or 25-OH-D₃-3-BE (13.2 μg, molar equivalent of 25-OH-D₃) or vehicle were administered i.p. in alternate days for two weeks. At the end of treatment, 25-OH-D₃-treated mice were very sick, and most of them died (as shown above, 3.3 μg/Kg of 25-OH-D₃ did not cause toxicity. Therefore observed toxicity could be due to a higher dose or tumor load or a combination of both). The 25-OH-D₃-3-BE-treated mice were viable and there was no significant loss of body weight. At the end, mice were anesthetized, tumors were removed, and blood was collected. Tumors were embedded in paraffin and sections were made.

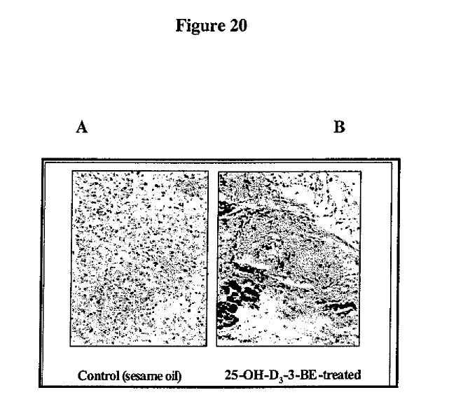

Hematoxylin-Eosin staining of the tumor sections showed that vehicle-treated tumor contained tightly packed growing cells (see, FIG. 20A). In contrast, the 25-OH-D₃-3-BE-treated tumors were considerably less cellular (see, FIG. 20B). This histological study clearly indicated that there was a large difference in the cellular structure in treated and untreated tumors.

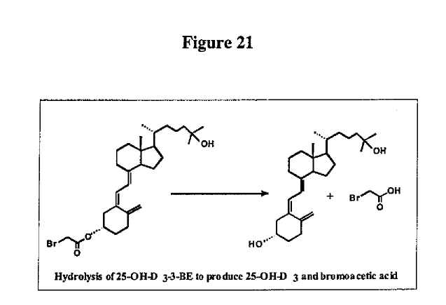

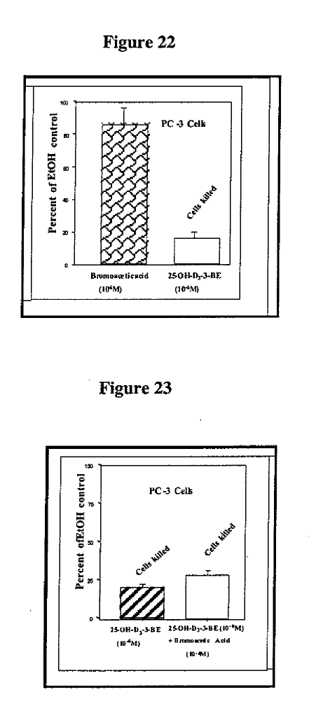

Possible mechanistic aspects of the observed antiproliferative and cytotoxic properties of 25-OH-D₃-3-BE have been considered. Without wishing to be bound by theory, possibly the random alkylation by 25-OH-D₃-3-BE or its hydrolyzed products could be involved. Since 25-OH-D₃-3-BE contains an ester bond, its hydrolysis by esterases in growing cells would produce equimolar amounts of bromoacetic acid (BAA) and 25-OH-D₃ (see, FIG. 21). BAA is a non-specific alkylating agent, and observed effects of 25-OH-D₃-3-BE could be due to either BAA or 25-OH-D₃ or a combination of two. As shown in FIG. 22, BAA (10⁻⁶M) was not antiproliferative to PC3 cells. A combination of 25-OH-D₃-3-BE and BAA produced similar growth-inhibitory effect as 25-OH-D₃-3-BE alone (see, FIG. 23). These results emphasized that the growth inhibitory effect of 25-OH-D₃-3-BE is related solely to its un-hydrolyzed form.

Furthermore, fetal calf serum (FCS) contains many proteins, including vitamin O-binding protein, which potentially could absorb 25-OH-D₃-3-BE before it reacts with VDR. Typically, the assays described herein were carried out in a media containing 10% FCS (after serum-depriving the cells for 15 hours). Therefore, these results strongly suggest that observed properties of 25-OH-D₃-3-BE are not due to random interaction with cellular proteins, and the effects are most probably due to intact form of 25-OH-D₃-3-BE.

An apoptotic mechanism of cytotoxicity by 25-OH-D₃-3-BE in pC-3 cells has also been considered.

As shown in FIG. 20, 25-OH-D₃-3-BE caused a complete change in the morphology of tumor cells that is often due programmed cell-death (apoptosis). This is most commonly manifested in the fragmentation of nuclear DNA producing characteristic ‘DNA-ladder’ in agarose gels.

(a) DNA-fragmentation analysis: PC-3 cells (2×10⁶) were treated with 250 nM of 1,25(OH)₂D₃, 25-OH-D₃ or 25-OH-D₃-3-BE for 10 hours. The cells were harvested and lysed in 0.5 ml lysis buffer (20 mM Tris-HCl, 10 mM EDTA, 0.5% Triton X-100, pH 8.0), and DNA was extracted using phenol-chloroform procedure. The DNA was re-suspended in 0.1 ml of 20 mM Tris-HCl, pH 8 and treated with Rnase, followed by electrophoresis on 1.2% agarose gel in TAE. The DNA was visualized under UV light after ethidinium bromide staining.

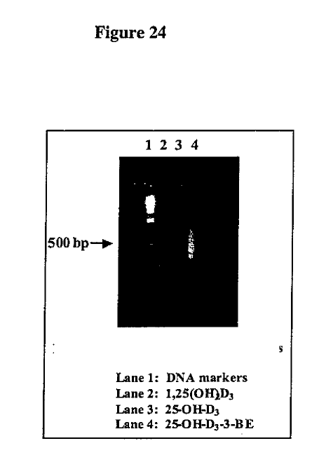

DNA fragmentation was observed in the case of 25-OH-D₃-3-BE 25-BE treated cells indicating apoptosis of PC-3 cells, where as no such DNA cleavage was observed in case of 1,25(OH)₂D₃, 25-OH-D₃ treated cells (see, FIG. 24).

Caspase-activity: There are several markers for apoptosis, and caspase activity is one of them; and their levels normally go up under apoptotic conditions. Investigators determined the levels of caspases 3, 8 and 9 by Caspase Colorimetric assay kits from R&D Systems (Minneapolis, Minn.) and used according to the manufacturer's instructions. Briefly, PC3 cells (1×10⁶) were treated with 10 nM of 1,25(OH)₂D₃, 25-OH-D₃ or 25-OH-D₃-3-BE for 14 hours in culture medium (DMEM, 10% FBS and antibiotics). The cells were collected by centrifugation at 1000 RPM for 5 min. The cell pellet was lysed with lysis buffer, and the lysate was incubated on ice for 10 min. and centrifuged for 10,000 RPM for 5 min. Protein was estimated using Bradford protein estimation kit (BioRad). The enzymatic reactions were carried out in 96 well plate. For each reaction 100 μg lysate protein in 50 μl was incubated with 50 μl of 2× reaction buffer and 5 μl of caspase-3 or caspase-8 or caspase-9 colorimetric substrate for 2 h at 37° C. The absorbance was determined at 405 nm.

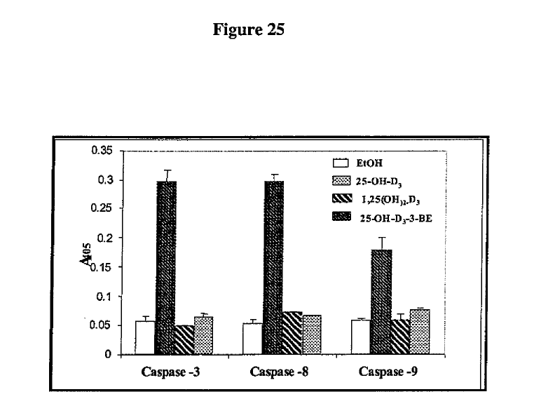

The results indicated that Caspase-3, 8 and 9 were induced in 25-OH-D₃-3-BE treated PC-3 cells in contrast to 1,25(OH)₂D₃ or 25-OH-D₃-treated cells (see, FIG. 25). Furthermore, these results strongly suggested that 25-OH-D₃-3-BE induced Caspase-dependent apoptosis in PC-3 cells.

The studies strongly suggest that 1,25(OH)₂D₃-3-BE and 25-OH-D₃-3-BE are strongly antiproliferative (cytostatic) towards prostate cells in general, but cytotoxic only to prostate cancer cells. These results also suggest that 25-OH-D₃-3-BE can be developed as a non-toxic vitamin D-analog for prostate cancer.

It is well known that many prostate cancer patients either do not respond to androgens or develop androgen-resistance, particularly in advanced stages. It is also established that 1,25(OH)₂D₃ and its metabolites and analogs derive their biological properties via the nuclear vitamin D receptor (VDR); and there is no evidence thus far to suggest that VDR is either absent or mutated (to be non-functional) in early, advanced or metastatic stages of prostate cancer. Hence, 1,25(OH)₂D₃-3-BE, 25-OH-D₃-3-BE and similar VDR-cross-linking analogs of 1,25(OH)₂D₃, which target VDR for their action, could be effective therapeutic agents for androgen-sensitive as well as androgen-refractory prostate cancer at all stages, including metastatic cancer. This hypothesis is supported by the observation that 1,25(OH)₂D₃-3-BE and 25-OH-D₃-3-BE appeared to be equally antiproliferative and cytotoxic to LNCaP (androgen-responsive) and PC3 (androgen-refractory) cells.

The invention is also directed to methods of using a vitamin D receptor specific binding agent that forms a covalent bond with the receptor as a treatment for cancer, e.g., prostate cancer. In one aspect, the therapeutic agent is an alkylating or acylating agent such as the vitamin D derivatives, 1α,25-dihydroxy-vitamin D₃-3β-(2)-bromoacetate and 25-hydroxy-vitamin D₃-3β-(2)-bromoacetate or one of their α-halocarbonyl or epoxide derivatives, which would similarly form a covalent bond with the hormone-binding pocket of the receptor.

In one embodiment, the invention is directed to methods of treating and/or preventing cancer in a subject by administering an effective amount of an analog of 1,25(OH)₂D₃. In one aspect, the analog to be administered is 1,25(OH)₂D₃-3-BE. In another aspect, the analog to be administered is 25(OH)₂D₃-3-BE. An “effective dose” is that dose of an agent (e.g., an analog of 1,25(OH)₂D₃) required to achieve a predetermined physiological effect, such as tumor size reduction, while not exceeding a subject's tolerance for the agent.

In another embodiment, the invention is directed to the treatment and/or prevention of cancer using combination therapy. In this embodiment, a subject is administered a combination of an effective amount of an analog of 1,25(OH)₂D₃. In one aspect, the analog to be administered is 1,25(OH)₂D₃-3-BE together with a known oncolytic agent. The administration of both components can be simultaneously, or in tandem.

The compositions of the present invention include antitumor drugs. Cycle-active agents are drugs that require a cell to be in cycle, i.e., actively going through the cell cycle preparatory to cell division to be cytotoxic. Some of these drugs are effective primarily against cells in one of the phases of the cell. The importance of this designation is that cell cycle-active agents are usually schedule-dependent, and that duration of exposure is as important and usually more important than dose. In contrast, noncell cycle-active agents are usually not schedule-dependent, and effects depend on the total dose administered, regardless of the schedule. Alkylating agents are generally considered to be noncycle active, whereas antimetabolites are prototypes of cycle-active compounds.

An example of cell cycle-active agents is fluoropyrimidines, such as 5-fluorouracil (5-FU) and 5-fluorodeoxyuridine (5-FUdR). 5-FU exerts its cytotoxic effects by inhibition of DNA synthesis, or by incorporation into RNA, thus inhibiting RNA processing and function. The active metabolite of 5-FU that inhibits DNA synthesis through potent inhibition of thymidylate synthase is 5-fluorodeoxyuridylate (5-FdUMP). In rapidly growing tumors, inhibition of thymidylate synthetase appears to be the key mechanism of cell death caused by 5-FU; however, in other tumors, cell death is better correlated with incorporation of 5-FU into RNA. Incorporation of 5-FU into DNA can occur also and may contribute to 5-FU cytotoxicity.

5-FU and 5-FUdR have antitumor activity against several solid tumors, most notably colon cancer, breast cancer, and head and neck cancer. A preparation containing 5-FU is used topically to treat skin hyperkeratosis and superficial basal cell carcinomas.

The major limiting toxicities of 5-FU and 5-FUdR include marrow and GI toxicity. Stomatitis and diarrhea usually occur 4-7 days after treatment. Further treatment is usually withheld until recovery from the toxic side-effects occurs. The nadir of leukopenia and of thrombocytopenia usually occurs 7-10 days after a single dose of a 5-day course. The dose-limiting toxicity to infusions of 5-FUdR through the hepatic artery is transient liver toxicity, occasionally resulting in biliary sclerosis. Less common toxicities noted with 5-FU after systemic administration are skin rash, cerebellar symptoms and conjunctivitis.

Another example of a cell cycle-active agent is methotrexate. This folate antagonist was one of the first antimetabolites shown to induce complete remission in children with ALL. Methotrexate (amethopterin) and aminopterin are analogs of the vitamin folic acid. Methotrexate, and similar compounds, acts by inhibiting the enzyme dihydrofolate reductase. As a consequence of this inhibition, intracellular folate coenzymes are rapidly depleted. These coenzymes are required for thymidylate biosynthesis as well as purine biosynthesis, as such, DNA synthesis is blocked by the use of methotrexate and alike. There is considerable toxicity associated with the use of methotrexate such as myelosuppression and GI distress. An early sign of methotrexate toxicity to the GI tract is mucositis. Severe toxicity can result in diarrhea that is due to small bowel damage that can progress to ulceration and bleeding.

Cytosine arabinoside (ara-C) is an antimetabolite analog of deoxycytidine. In the analog, the OH group is in the β configuration at the 2′ position. This compound was first isolated from the sponge Cryptothethya crypta. Ara-C is the drug of choice for the treatment of acute myelocytic leukemia. Ara-C is converted intracellularly to the nucleotide of triphosphate (ara-CTP) that is both an inhibitor of DNA polymerase and incorporated into DNA. The latter event is considered to cause the lethal action of ara-C. Nausea and vomiting are observed with patients being treated with ara-C.

There is a myriad of other chemotherapeutics considered to be within the scope of this invention. Purine analogs, such as 6-mercaptopurine and 6-thioguanine, define drugs that are also employed in the war against cancer. Hydroxyurea is another drug that is used to treat cancer. Hydroxyurea inhibits ribonucleotide reductase, the enzyme that converts ribonucleotides at the diphosphate level to deoxyribonucleotides. Vinca alkaloids are also involved in the treatment of cancer. The vinca alkaloids include vinblastine, vincristine, and vindesine. Epipodophyllotoxin is a derivative of podophyllotoxin that is used in the treatment of such cancers as leukemia, Hodgkin's, and other cancers.

Alkylating agents such as mechlorethamine, phenylalanine mustard, chlorambucil, ethylenimines and methyl melamines, and alkylsulfonates are employed to treat various cancers.

Nitrosoureas like carmustine, lomustine, and streptozocin are used to treat various cancers and have the ability to readily cross the blood-brain barrier.

Cisplatin (diamino-dichloro-platinum) is a platinum coordination complex that has a broad spectrum antitumor activity. Cisplatin is a reactive molecule and is able to form inter- and intrastrand links with DNA in order to cross-link proteins with the DNA. Carboplatin is another platinum based antitumor drug.

Triazenes like dacarbazine and procarbazine are apart of the antitumor arsenal.

There are antibiotics that have antitumor activity such as anthracyclines, such as doxorubicin, daunorubicin, and mitoxantrone. Other antitumor antibiotics include bleomycin, dactinomycin, mitomycin C, and plycamycin.

There are other antitumor drugs, like asparaginase, that are considered to be within the scope of this invention. These and the other drugs mentioned above all have a toxicity profile that is well known to those skilled in the art.

Other therapeutic agents that can be used in the present invention include cyclophosphamide (cytoxan), melphalan (alkeran), chlorambucil (leukeran), carmustine (BCNU), thiotepa, busulfan (myleran); glucocorticoids such as prednisone/prednisolone, triamcinolone (vetalog); other inhibitors of protein/DNA/RNA synthesis such as dacarbazine (DTIC), procarbazine (matulane); and paclitaxel.

Within a particular embodiment of the present invention, the therapeutic agent is paclitaxel, a compound that disrupts microtubule formation by binding to tubulin to form abnormal mitotic spindles. Briefly, paclitaxel is a highly derivatized diterpenoid (Wani et al., J. Am. Chem. Soc. 93:2325, 1971, the entire teaching of which is incorporated herein by reference) which has been obtained from the harvested and dried bark of Taxus brevifolia (Pacific Yew) and Taxomyces Andreanae and Endophytic Fungus of the Pacific Yew (Stierle et al., Science 60:214-216, 1993, the entire teaching of which is incorporated herein by reference).

“Paclitaxel” (which should be understood herein to include prodrugs, analogues and derivatives such as, for example, TAXOL®, TAXOTERE®, Docetaxel, 10-desacetyl analogues of paclitaxel and 3′N-desbenzoyl-3′N-t-butoxy carbonyl analogues of paclitaxel) can be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11): 1580-1583, 1994; J. Am. Chem. Soc. 110:6558-6560, 1988, the entire teachings of which are incorporated herein by reference), or obtained from a variety of commercial sources, including for example, Sigma Chemical Co., St. Louis, Mo. (T7402—from Taxus brevifolia).

Representative examples of such paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′, 7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotal, 10-desacetoxy-7-deoxy-9-deoxotaxol, derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol format; 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′ succinyltaxol; 2′-(beta-alanyl)-taxol); 2′γ-amino-butyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′ orthocarboxy-benzoyl taxol; 2′aliphatic carboxylic acid derivatives of taxol, Prodrugs {2′(N,N-diethylamino-propionyl)taxol, 2′(N,N-dimethyglycyl)taxol, 7(N,N-dimethyl-glycyl)taxol, 2′,7-di-(N,N-dimethylglycyl)taxol, 7(N,N-diethylaminopropionyl)taxol, 2′,7-di(N,N-diethyl-aminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-iso-leucyl)taxol, 2′-(L-valyl)taxol, 7-(L-valyl)taxol, 27-di(L-valyl)taxol, 2′-(L-phenylalanyl)taxol, 7-(L-phenylalany)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-de-acetyltaxol, and taxanes (e.g., baccatin III, cephalomannine, 10-deacetylbaccatin III, brevifoliol, yunantaxusin and taxusin).

Representative examples of microtubule depolymerizing (or destabilizing or disrupting) agents include Nocodazole (Ding et al., J. Exp. Med. 171(3):715-727, 1990; Dotti et al., J. Cell Sci. Suppl. 15:75-84, 1991; Oka et al., Cell Struct Funct. 16(2): 125-134, 1991; Wiemer et al., J. Cell. Biol. 136(1):71-80, 1997, the entire teachings of which are incorporated herein by reference); Cytochalasin B (Illinger et al., Biol. Cell 73(2-3):131-138, 1991, the entire teaching of which is incorporated herein by reference); Vinblastine (Ding et al., J. Exp. Med. 171(3):715-727, 1990; Dirk et al., Neurochem. Res. 15(11):1135-1139, 1990; Klinger et al., Biol. Cell 73(2-3):131-138, 1991; Wiemer et al., J. Cell. Biol. 136(1):71-80, 1997, the entire teachings of which are incorporated herein by reference); Vincristine (Dirk et al., Neurochem. Res. 15(11): 1135-1139, 1990; Ding et al., J. Exp. Med. 171(3):715-727, 1990, the entire teaching of which is incorporated herein by reference); Colchicine (Allen et al., Am. J. Physiol. 261(4 Pt. 1):L315-L321, 1991; Ding et al., J. Exp. Med. 171(3):715-727, 1990; Gonzalez et al., Exp. Cell. Res. 192(1):10-15, 1991; Stargell et al., Mol. Cell. Biol. 12(4):1443-1450, 1992, the entire teachings of which are incorporated herein by reference); CI-980 (colchicine analogue) (Garcia et al., Anticancer Drugs 6(4):533-544, 1995, the entire teaching of which is incorporated herein by reference); Colcemid (Barlow et al., Cell. Motil. Cytoskeleton 19(1):9-17, 1991; Meschini et al., J. Microsc. 176(Pt. 3):204-210, 1994; Oka et al., Cell Struct. Funct. 16(2):125-134, 1991, the entire teachings of which are incorporated herein by reference); Podophyllotoxin (Ding et al., J. Exp. Med. 171(3):715-727, 1990, the entire teaching of which is incorporated herein by reference); Benomyl (Hardwick et al., J. Cell. Biol. 131(3):709-720, 1995; Shero et al., Genes Dev. 5(4):549-560, 1991, the entire teachings of which are incorporated herein by reference); Oryzalin (Stargell et al., MeI. Cell. Biol. 12(4): 1443-1450, 1992, the entire teaching of which is incorporated herein by reference); Majusculamide C (Moore, J. Ind. Microbiol. 16(2):134-143, 1996, the entire teaching of which is incorporated herein by reference); Demecolcine (Van Dolah and Ramsdell, J. Cell. Physiol. 166(1):49-56, 1996; Wiemer et al., J. Cell. Biol. 136(1):71-80, 1997, the entire teaching of which is incorporated herein by reference); and Methyl-2-benzimidazolecarbamate (MBC) (Brown et al., J. Cell. Biol. 123(2):387-403, 1993, the entire teaching of which is incorporated herein by reference).

Any of the identified compounds of the present invention can be administered to a subject, including a human, by itself, or in pharmaceutical compositions where it is mixed with suitable carriers or excipients at doses therapeutically effective to prevent, treat or ameliorate a variety of disorders, including those characterized by that outlined herein. A therapeutically effective dose further refers to that amount of the compound sufficient result in the prevention or amelioration of symptoms associated with such disorders. Techniques for formulation and administration of the compounds of the instant invention may be found in Goodman and Gilman's The Pharmacological Basis of Therapeutics, Pergamon Press, latest edition.

The compounds of the present invention can be targeted to specific sites by direct injection into those sites. Compounds designed for use in the central nervous system should be able to cross the blood-brain barrier or be suitable for administration by localized injection.

Pharmaceutical compositions suitable for use in the present invention include compositions wherein the active ingredients are contained in an effective amount to achieve its intended purpose. More specifically, a therapeutically effective amount means an amount effective to prevent development of or alleviate the existing symptoms and underlying pathology of the subject being treated. Determination of the effective amounts is well within the capability of those skilled in the art.

For any compound used in the methods of the present invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that results in the attenuation of symptoms or a prolongation of survival in a subject. Toxicity and therapeutic efficacy of such compounds can be determined by standard pharmaceutical procedures in cell cultures or experimental animals, e.g., for determining the LD₅₀ (the dose lethal to 50% of a given population) and the ED₅₀ (the dose therapeutically effective in 50% of a given population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of a patient's condition. Dosage amount and interval can be adjusted individually to provide plasma levels of the active moiety that are sufficient to maintain the desired effects.

In case of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions of the present invention can be manufactured in a manner that is itself known, e.g., by means of conventional mixing, dissolving, granulating, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries, which facilitate processing of the active compounds into preparations that can be used pharmaceutically. Proper formulation is dependent upon the route of administration chosen.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinyl-pyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodi-fluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage for, e.g., in ampoules or in multidose containers, with added preservatives. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspension. Suitable lipohilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations previously described, the compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

A pharmaceutical carrier for the hydrophobic compounds of the invention is a co-solvent system comprising benzyl alcohol, a non-polar surfactant, a water-miscible organic polymer, and an aqueous phase. Naturally, the proportions of a co-solvent system can be varied considerably without destroying its solubility and toxicity characteristics. Furthermore, the identity of the co-solvent components can be varied.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known to those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration can, e.g., include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, e.g., via injection of the compound directly into an affected area, often in a depot or sustained release formulation.

Furthermore, one can administer the compound in a targeted drug delivery system, e.g., in a liposome coated with an antibody specific for affected cells. The liposomes will be targeted to and taken up selectively by the cells.

The compositions can, if desired, be presented in a pack or dispenser device that can contain one or more unit dosage forms containing the active ingredient. The pack can, e.g., comprise metal or plastic foil, such as a blister pack. The pack or dispenser device can be accompanied by instruction for administration. Compositions comprising a compound of the invention formulated in a compatible pharmaceutical carrier can also be prepared, placed in an appropriate container, and labeled for treatment of an indicated condition. Suitable conditions indicated on the label can include treatment of a disease such as described herein.

Although the invention has been described with respect to various embodiments, it should be realized this invention is also capable of a wide variety of further and other embodiments within the spirit and scope of the appended claims.

What is claimed is:

1. A composition comprising an analog of 1,25(OH)₂D₃, wherein said analog is alkylated or acylated.

2. The composition of claim 1, wherein said analog cross links said 1,25(OH)₂D₃ to a hormone-binding pocket of VDR.

3. The composition of claim 1, wherein said analog is selected from the group consisting of 1,25(OH)₂D₃-3-BE, 25(OH)₂D₃-3-BE and derivatives thereof.

4. A method of treating and/or preventing cancer in a subject by administering to said subject an effective amount of an analog of 1,25(OH)₂D₃, wherein said analog is alkylated or acylated.

5. The method of claim 4, wherein said cancer is prostate cancer.

6. The method of claim 4, wherein said analog cross links said 1,25(OH)₂D₃ to a hormone-binding pocket of VDR.

7. The method of claim 4, wherein said analog is selected from the group consisting of 1,25(OH)₂D₃-3-BE, 25(OH)₂D₃-3-BE and derivatives thereof.

8. A method of treating and/or preventing cancer in a subject by administering a combination pharmaceutical formulation comprising an effective amount of an analog of 1,25(OH)₂D₃ and an effective amount of a known oncolytic agent, wherein said analog is alkylated of acylated.

9. The method of claim 8, wherein said cancer is prostate cancer.

10. The method of claim 8, wherein said analog cross links said 1,25(OH)₂D₃ to a hormone-binding pocket of VDR.

11. The method of claim 8, wherein said analog is selected from the group consisting of 1,25(OH)₂D₃-3-BE, 25(OH)₂D₃-3-BE and derivatives thereof.

12. The method of claim 8, wherein said oncolytic agent is selected from the group consisting of paclitaxel, 5-FU, 5-FUdR, methotrexate, ara-C, 6-mercaptopurine, 6-thioguanine, hydroxyurea, mechlorethamine, phenylalanine mustard, chlorambucil, ethylenimines, methyl melamines, carmustine, lomustine, streptozocin, Cisplatin, Carboplatin, dacarbazine, procarbazine, doxorubicin, daunorubicin, mitomycin C, plycamycin, cyclophosphamide, melphalan, chlorambucil, carmustine, thiotepa, busulfan, prednisone, prednisolone, triamcinolone, and derivatives thereof.
